(19) United States
(12) Patent Application Publication
(10) Pub. No.: US 2015/0297660 A1
(43) Pub. Date: Oct. 22, 2015

(54) Magnolia officinalis Rehd. et Wils. extract feed additive and preparation method thereof

(71) Applicant: Shanghai Zhao Xiang Biological Technology Co., LTD, Shanghai (CN)

(72) Inventors: Jiaming Chen, Shanghai (CN); Xuemin Chen, Shanghai (CN); Xuefeng Chen, Shanghai (CN); Danrong Zheng, Shanghai (CN); Huizhen Chen, Shanghai (CN); Shasha Chen, Shanghai (CN); Ling Lu, Shanghai (CN)

(73) Assignee: Shanghai Zhao Xiang Biological Technology Co., LTD

(21) Appl. No.: 14/689,024

(22) Filed: Apr. 16, 2015

(30) Foreign Application Priority Data
Apr. 16, 2014 (CN) 201410153229.9

(51) Int. Cl. A61K 36/575; A61K 47/36; A61K 47/46; A23L 1/30; A61K 47/02

(52) U.S. Cl. CPC A61K 36/575; A23L 1/3002; A23L 1/301; A61K 47/02; A61K 47/46; A61K 47/36; A23V 2002/00

(57) ABSTRACT

A Magnolia officinalis Rehd. et Wils. extract feed additive and a preparation method thereof are provided. The Magnolia officinalis Rehd. et Wils. extract feed additive takes a Magnolia officinalis Rehd. et Wils. extract and a carrier as raw materials. The Magnolia officinalis Rehd. et Wils. extract is prepared by the ultrasonic auxiliary water extraction. The preparation method of the present invention has less raw material waste, high activity component content in the extract and acquisition rate of the extractum, which facilitates improving the effect efficiency and reducing production cost. The Magnolia officinalis Rehd. et Wils. extract feed additive as a natural plant feed additive has a better rehabilitating effect on gastric ulcer and anti-cancer of livestock and greatly reduces the economic loss due to gastric ulcer.

### CROSS REFERENCE OF RELATED APPLICATION

The present invention claims priority under 35 U.S.C. 119(a-d) to CN 201410153229.9, filed Apr. 16, 2014.

### BACKGROUND OF THE PRESENT INVENTION

1. Field of Invention

The present invention relates to a Chinese herbal medicine feed field, and more particularly to a Magnolia officinalis Rehd. et Wils. extract feed additive and a preparation method thereof.

2. Description of Related Arts

Plant extract is one of main feed antibiotic substitutes and is a natural substance. It is capable of promoting animal growth, improving animal constitution, increasing metabolism, improving production performance, resisting stress, preventing diseases, etc., which is in the leading level in China. Foreign countries have very strict requirements for food, and especially, European Union which is internationally the earliest, largest and most strict region where feed antibiotic is strictly prohibited. Feed additives are successfully prohibited in European Union, the reason is that: besides excellent feeding and management supporting facilities, there is a very important physical measurement that feed antibiotic is replaced by the plant extract (Chinese herbal medicine). The Chinese herbal medicine plays a very important role in successfully prohibiting feed antibiotic.

With the transformation of Chinese animal husbandry from traditional extensive to modern intensive production and management, and the increasing improvement of quality requirements to animal foods, it is an inevitable trend to replace feed antibiotics with Chinese herbal medicine feed additive with some advantages of avoiding disease, health care, promoting growing, no tolerance, no toxic effects and no residue. In China, green and safe agriculture has a unique advantage, and its market is vast and not vigorously mined. Therefore, it has huge market capacity, huge potential and huge space for development.

It is well known that diseases of pigs are various, and especially, the most severe is gastritis. If gastrointestinal tract has more severe malfunction, smooth muscle relaxation exercise is decreased, thus feeling swelling, or smooth muscle is spasmodically contracted, thus feeling serious stomachache, which are regarded as syndrome of stagnation by traditional Chinese medicine. In this case, breaking is generally better than regulating, promoting circulation, promoting flow, descending and depressing.

Magnolia officinalis Rehd. et Wils. is acrid in taste, warm in nature, and is capable of promoting circulation, resolving dampness, being warm in the pain, lowering the adverse flow and relieving asthma. Magnolia officinalis Rehd. et Wils. decoctum has a strong antibacterial effect on Staphylococcus, Streptococcus, Dysentery Bacillus, Pasteurella and Vibrio Cholerae, and has a relaxation effect on striated muscle stiffness. Currently, traditional Chinese medicine is commonly used in China, long-term application practice shows that the traditional Chinese medicine is safe and efficient, and has a large development value and research significance. However, related researches on the value and function of Magnolia officinalis Rehd. et Wils. as fodder have not been reported.

### SUMMARY OF THE PRESENT INVENTION

An object of the present invention is to overcome the shortcomings of the above prior art and provide a Magnolia officinalis Rehd. et Wils. extract feed additive and a preparation method thereof. While extracting a traditional Chinese medicine plant, the most critical aspect is to select the solvent, set the steps, optimize the temperature and set ratios of components. It is found through a large number of experimental studies that the present invention achieves a most efficient, simple and practical method of preparing a Magnolia officinalis Rehd. et Wils. extract without any pollutions, and extracts farthest active ingredients from the Magnolia officinalis Rehd. et Wils. extract for preparing a natural plant feed additive to have a better rehabilitating effect on gastric ulcer and anti-cancer of livestock and greatly reduce the economic loss due to gastric ulcer.

The above object is achieved by the technical solution as follows.

On the one hand, the present invention relates to a Magnolia officinalis Rehd. et Wils. extract feed additive comprising a Magnolia officinalis Rehd. et Wils. extract and a carrier, wherein the Magnolia officinalis Rehd. et Wils. extract is prepared by an ultrasonic auxiliary water extraction.

Preferably, a weight ratio of the Magnolia officinalis Rehd. et Wils. extract and the carrier is 1:(2-3).

Preferably, the carrier is a mixture of attapulgite, and pine needle with a weight ratio of 1:(1-2).

On the other hand, the present invention also relates to a method of preparing the above Magnolia officinalis Rehd. et Wils. extract feed additive, comprising steps of:

(1) pretreating and smashing Magnolia officinalis Rehd. et Wils., adding ethanol solution to immerse for 4-5 h; removing to an ultrasonic extractor for ultrasonically extracting at room temperature for 30-40 min; filtering after the extraction for obtaining a filtrate and a first solid residue; vacuum evaporating for removing ethanol in the filtrate to obtain a first extracting solution;

(2) adding eight times an amount of water into the first solid residue, boiling for 4-6 h at a steam pressure of 0.5-0.7 MPa, filtering to obtain a second extracting solution and a second solid residue;

(3) adding six times an amount of water into the second solid residue, boiling for 2-4 h at a steam pressure of 1.5-1.6 MPa, filtering to obtain a third extracting solution and a third solid residue;

(4) mixing the first extracting solution, the second extracting solution and the third extracting solution, concentrating for obtaining a first extractum; spraying and drying to obtain a Magnolia officinalis Rehd. et Wils. extract dry powder; and (5) evenly mixing the Magnolia officinalis Rehd. et Wils. extract dry powder with a carrier to obtain the Magnolia officinalis Rehd. et Wils. extract feed additive.

Preferably, the step of pretreating and smashing Magnolia officinalis Rehd. et Wils. comprises naturally drying and impurity removing stems and leafs of the Magnolia officinalis Rehd. et Wils., drying for 6 h at 60° C., smashing, and sieving through a sieve with an aperture of 1.0-1.2 mm. If a particle is undersize, the Magnolia officinalis Rehd. et Wils. powder is easy to float on the feed liquid, so that the extraction effect is affected; if the particle is oversize, the extraction efficiency is reduced more.

Preferably, an amount of the added ethanol solution is 15-20 ml ethanol solution per gram Magnolia officinalis Rehd. et Wils., wherein a concentration of ethanol in the ethanol solution is 75 wt %-85 wt %. The concentration and amount of the ethanol solution have larger effects on the acquisition rate of the extractum. When the amount of the ethanol solution is increased to a certain degree (wherein a concentration of ethanol solution is 80 wt %, an amount thereof is that 20 ml ethanol solution is added per gram Magnolia officinalis Rehd. et Wils.), increasing the amount of the ethanol solution has no obvious effects on the acquisition rate of the extractum. After overall consideration of cost and effect, 15-20 ml ethanol solution is added per gram Magnolia officinalis Rehd. et Wils., wherein when a concentration of ethanol in the ethanol solution is 75 wt %-80 wt %, the technology is best.

Preferably, while ultrasonically extracting, an ultrasonic frequency is 125-130 KHz.

Preferably, while ultrasonically extracting, moisture is distilled simultaneously for collecting a distillate. The distillate is Magnolia officinalis Rehd. et Wils. volatile oil containing eudesmol and its isomers, and P-cymene. According to the present invention, the distillate is sold as a secondary product and is capable of mixing with the first extraction solution, the second extraction solution and the third extraction solution, and then concentrating for subsequent preparation.

Preferably, in the step (2), acetic acid is added to adjust a PH value of solution to 6.5, and in the step (3), saturated NaOH is added to adjust a PH value of solution to 8.5.

Preferably, before spraying and drying, the step (4) further comprises steps of: eluting the first extractum with silica gel column chromatography gradient which takes petroleum ether/ethyl acetate as an eluent with a ratio of petroleum ether/ethyl acetate=10:1 and 5:1, and an eluting time of 15 min, combining eluting solutions, concentrating for obtaining a second extractum, spraying and drying the second extractum to obtain the Magnolia officinalis Rehd. et Wils. extract dry powder.

Compared with the prior art, the present invention has beneficial effects as follows.

(1) It has a better rehabilitating effect on gastric ulcer and anti-cancer of livestock.

(2) It is capable of avoiding comprehensive complicating diseases due to porcine reproductive and respiratory syndrome (PRRS), eperythrozoonosis and toxoplasmosis.

(3) It has strong palatability and is capable of improving feed intake and promoting growing.

(4) It is natural and green without any chemical medicines or hormone.

These and other objectives, features, and advantages of the present invention will become apparent from the following detailed description, and the appended claims.

### DETAILED DESCRIPTION OF THE PREFERRED EMBODIMENT

The present invention is further explained in detail with the accompanying embodiments.

### Example 1

The example 1 relates to a Magnolia officinalis Rehd. et Wils. extract feed additive. A method of preparing the above Magnolia officinalis Rehd. et Wils. extract feed additive comprises steps of:

Step 1: naturally drying and impurity removing stems and leafs of Magnolia officinalis Rehd. et Wils., drying for 6 h at 60° C., smashing, sieving through a sieve with an aperture of 1.2 mm, adding 20 ml, 80 wt % ethanol solution per gram Magnolia officinalis Rehd. et Wils. to immerse for 6 h; removing to an ultrasonic extractor for ultrasonically extracting at room temperature and a frequency of 125 KHz for 35 mM; filtering after the extraction for obtaining a filtrate and a first solid residue; vacuum evaporating for removing ethanol in the filtrate to obtain a first extracting solution;

Step 2: adding eight times an amount of water into the first solid residue, boiling for 5 h at a steam pressure of 0.7 MPa, filtering to obtain a second extracting solution and a second solid residue;

Step 3: adding six times an amount of water into the second solid residue, boiling for 3 h at a steam pressure of 1.6 MPa, filtering to obtain a third extracting solution and a third solid residue;

Step 4: mixing the first extracting solution, the second extracting solution and the third extracting solution, concentrating till D (which is a proportion of the mixed solution)=0.6-1.2 for obtaining an extractum; putting the extractum into a spray dryer with an inlet air temperature of 180° C. and an outlet air temperature of 80° C. (which is able to be in a range of 70-90° C.) to obtain a Magnolia officinalis Rehd. et Wils. extract dry powder; and

Step 5: evenly mixing the Magnolia officinalis Rehd. et Wils. extract dry powder with a carrier (which is a mixture of attapulgite and pine needle powder with a weight ratio of 1:1) to obtain the Magnolia officinalis Rehd. et Wils. extract feed additive, wherein a weight ratio of the Magnolia officinalis Rehd. et Wils. extract dry powder and the carrier is 1:3.

### Comparative Example 1

The comparative example 1 relates to a Magnolia officinalis Rehd. et Wils. extract feed additive. A method of preparing the above Magnolia officinalis Rehd. et Wils. extract feed additive comprises steps of:

Step 1: naturally drying and impurity removing stems and leafs of Magnolia officinalis Rehd. et Wils., drying for 6 h at 60° C., smashing, sieving through a sieve with an aperture of 1.2 mm; adding the sieved Magnolia officinalis Rehd. et Wils. into an alcohol extraction tank, adding eight times an amount of 80 wt % ethanol, boiling for 5 h at a steam pressure of 0.7 MPa, making an ethanol reflux; filtering after firstly extracting ethanol for obtaining a first filtrate and a first filter residue, wherein ethanol is recycled from the first filtrate by a decompression concentration tank till a concentration of the recycled ethanol is smaller than 60%, concentrating the first filtrate to obtain a concentrated solution;

Step 2: adding eight times an amount of 80 wt % ethanol into the first filter residue, boiling for 5 h at a steam pressure of 0.7 MPa, making an ethanol reflux, wherein ethanol is recycled till a concentration of the recycled ethanol is smaller than 60%, filtering to obtain a second filtrate and a second filter residue;

Step 3: adding six times an amount of water into the second filter residue, boiling for 3 h at a steam pressure of 1.6 MPa, filtering to obtain a third filtrate and a third filter residue; discarding the third filter residue;

Step 4: mixing the concentrated solution, the second filtrate and the third filtrate, concentrating till D (which is a proportion of the mixed solution)=0.6-1.2 for obtaining an extractum; putting the extractum into a spray dryer with an inlet air temperature of 180° C. and an outlet air temperature of 80° C. to obtain a Magnolia officinalis Rehd. et Wils. extract dry powder; and

Step 5: evenly mixing the Magnolia officinalis Rehd. et Wils. extract dry powder with a carrier (which is a mixture of attapulgite and pine needle powder with a weight ratio of 1:1) to obtain the Magnolia officinalis Rehd. et Wils. extract feed additive, wherein a weight ratio of the Magnolia officinalis Rehd. et Wils. extract dry powder and the carrier is 1:3.

### Example 2

An object of the example 2 is to study the effects of a PH value in a step of water extraction after ultrasonically extracting on a ratio of dry extraction and an extraction ratio of active components. The preparation method of the example 2 is as same as that of the example 1, and the differences therebetween are that: in the step (2) of the example 2, respectively add acetic acid to adjust a PH value of solution to 5.5, 6.0 and 6.5, and respectively add saturated NaOH to adjust a PH value of solution to 8.0, 8.5 and 9.0 in the step (3) of the example 2 for forming nine experimental groups, wherein nine experimental groups 2a-2i respectively correspond to nine groups of PH values in step (2) and step (3) which are respectively 5.5, 8.0; 5.5, 8.5; 5.5, 9.0; 6.0, 8.0; 6.0, 8.5; 6.0, 9.0; 6.5, 8.0; 6.5, 8.5; 6.5, 9.0.

Take 100 g Magnolia officinalis Rehd. et Wils. medicinal material to make a comparative experiment on the example 1, the example 2 and the comparative example 1 as follows.

Measure the ratio of dry extraction: respectively mix the first extracting solution, the second extracting solution and the third extracting solution obtained by the above nine experimental groups and the example 1; the concentrated solution, the second filtrate and the third filtrate obtained by the comparative example 1, and concentrate the mixture to be with a volume of 500 ml, take 20 ml concentrated solution to a moisture analyzer for determining moisture, and calculate the ratio of dry extraction of the extract based on a moisture value.

Measurement results are shown in Table 1.

TABLE 1

| | EG | EG | EG | EG | EG | EG | EG | EG | EG | | |
|---|---|---|---|---|---|---|---|---|---|---|---|
| | 2a | 2b | 2c | 2d | 2e | 2f | 2g | 2h | 2i | E1 | CE1 |
| RODE | 22.1 | 22.7 | 22.6 | 23.2 | 22.5 | 23.0 | 22.9 | 25.8 | 22.7 | 23.1 | 18.7 |
| (%) | | | | | | | | | | | |

* Note: Experimental Group is abbreviated as EG, Example 1 is abbreviated as E1, Comparative Example is abbreviated as CE1, and the ratio of dry extraction is abbreviated as RODE.

It can be seen from Table 1 that when acetic acid is added to adjust the PH value of solution to 6.5 in the step (2), and saturated NaOH is added to adjust the PH value of solution to 8.5 in the step (3), the ratio of dry extraction of the extract is best.

### Example 3

The example 3 relates to a Magnolia officinalis Rehd. et Wils. extract feed additive, the preparation method thereof is as same as that of the example 1 and the differences therebetween are as follows.

Before spraying drying, following steps further included: eluting the extractum with silica gel column chromatography gradient which takes petroleum ether/ethyl acetate as an eluent with a ratio of petroleum ether/ethyl acetate=10:1; 5:1; 3:1; 2:1; 1:1, 1:5 and an eluting time of 15 min, respectively collecting eluting solution, concentrating for obtaining a new extractum, spraying and drying to obtain the Magnolia officinalis Rehd. et Wils. extract dry powder. Evenly mix the six groups of Magnolia officinalis Rehd. et Wils. extract dry powder with a carrier (which is a mixture of attapulgite and pine needle powder with a weight ratio of 1:1) to obtain the Magnolia officinalis Rehd. et Wils. extract feed additives which are respectively recorded as Experimental group 3a, Experimental group 3b, Experimental group 3c, Experimental group 3d, Experimental group 3e and Experimental group 3f, wherein a weight ratio of the Magnolia officinalis Rehd. et Wils. extract dry powder and the carrier is 1:3.

### Example 4

An object of the example 4 is to study the effects of various carriers on the Magnolia officinalis Rehd. et Wils. extract feed additive. The preparation method thereof is as same as that of example 1 and the differences therebetween are as follows. The carriers are respectively selected from attapulgite, pine needle powder, maltodextrin, a mixture of attapulgite and pine needle powder with a weight ratio of 1:2, and a mixture of attapulgite and maltodextrin with a weight ratio of 1:2, which are respectively recorded as Experimental group 4a, Experimental group 4b, Experimental group 4c, Experimental group 4d and Experimental group 4e.

Comparatively experiment on the examples 1, 3, 4 and the comparative example 1 at the experimental place of Yuanheng animal Chinese medicine research center, Shanghai of China. The experimental animals are three-breed growing-finishing pigs bred in the late and fed with mixing materials, wherein 650 g Magnolia officinalis Rehd. et Wils. extract feed additives are added per ton fodder. No Magnolia officinalis Rehd. et Wils. extract feed additive is added to the comparative group. Experimental results are shown in Table 2.

TABLE 2

| | | | Average | Feed | Rate of | Incidence |
|---|---|---|---|---|---|---|
| | | Daily gain | daily feed | conversion | survival | of gastric |
| Test indexes | | (g) | intake (g) | ratio | (%) | ulcer (%) |
| Comparative | | 770 | 2364 | 22.01 | 97 | 32 |
| Group | | | | | | |
| Comparative | | 801 | 2342 | 5.87 | 98 | 27 |
| Example 1 | | | | | | |
| Example | 1 | 845 | 2271 | 2.6 | 100 | 22 |
| | 2h | 852 | 2259 | 2.55 | 100 | 20 |
| | 3a | 862 | 2243 | 2.45 | 100 | 12 |
| | 3b | 863 | 2248 | 2.42 | 100 | 13 |
| | 3c | 852 | 2268 | 2.81 | 99.5 | 20.5 |
| | 3d | 842 | 2273 | 2.76 | 99 | 21.8 |
| | 3e | 843 | 2269 | 2.75 | 98.5 | 22 |
| | 3f | 840 | 2275 | 2.69 | 98 | 23 |
| | 4a | 810 | 2300 | 3.10 | 99 | 24.5 |
| | 4b | 851 | 2279 | 2.61 | 99.5 | 23 |
| | 4c | 805 | 2310 | 3.12 | 98.5 | 26 |
| | 4d | 844 | 2272 | 2.58 | 100 | 20.5 |
| | 4e | 811 | 2302 | 3.05 | 99 | 25 |

It can be seen from Table 2 by comparing the examples 1, 4a, 4b, 4c, 4d and 4e that: the mixture of attapulgite and pine needle powder with a weight ratio of 1:(1-2) as the carrier is capable of playing a synergic role in the Magnolia officinalis Rehd. et Wils. extract and reducing the incidence of gastric ulcer of pigs. It can be found by comparing experimental groups in the example 3 that: when the eluent with the ratio of petroleum ether/ethyl acetate=10:1 and 5:1 is used, the eluted component is capable of significantly reducing the incidence of gastric ulcer of pigs and improving the rate of survival thereof. All in all, the Magnolia officinalis Rehd. et Wils. extract feed additive of the present invention is capable of enhancing the animal's immunity function and resistance to disease, reducing the incidence of gastric ulcer of pigs, having a better rehabilitating effect on gastric ulcer and anti-cancer of livestock and improving the animal's survival rate.

One skilled in the art will understand that the embodiment of the present invention as shown in the drawings and described above is exemplary only and not intended to be limiting.

It will thus be seen that the objects of the present invention have been fully and effectively accomplished. Its embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A Magnolia officinalis Rehd. et Wils. extract feed additive, comprising a Magnolia officinalis Rehd. et Wils. extract and a carrier, wherein the Magnolia officinalis Rehd. et Wils. extract is prepared by an ultrasonic auxiliary water extraction.

2. The Magnolia officinalis Rehd. et Wils. extract feed additive, as recited in claim 1, wherein a weight ratio of the Magnolia officinalis Rehd. et Wils. extract and the carrier is 1:(2-3).

3. The Magnolia officinalis Rehd. et Wils. extract feed additive, as recited in claim 1, wherein the carrier is a mixture of attapulgite and pine needle powder with a weight ratio of 1:(1-2).

4. A method of preparing a Magnolia officinalis Rehd. et Wils. extract feed additive, comprising steps of:
(1) pretreating and smashing Magnolia officinalis Rehd. et Wils., adding ethanol solution to immerse for 4-5 h; removing to an ultrasonic extractor for ultrasonically extracting at room temperature for 30-40 min; filtering after the extraction for obtaining a filtrate and a first solid residue; vacuum evaporating for removing ethanol in the filtrate to obtain a first extracting solution;
(2) adding eight times an amount of water into the first solid residue, boiling for 4-6 h at a steam pressure of 0.5-0.7 MPa, filtering to obtain a second extracting solution and a second solid residue;
(3) adding six times an amount of water into the second solid residue, boiling for 2-4 h at a steam pressure of 1.5-1.6 MPa, filtering to obtain a third extracting solution and a third solid residue;
(4) mixing the first extracting solution, the second extracting solution and the third extracting solution, concentrating for obtaining a first extractum; spraying and drying to obtain a Magnolia officinalis Rehd. et Wils. extract dry powder; and
(5) evenly mixing the Magnolia officinalis Rehd. et Wils. extract dry powder with a carrier to obtain the Magnolia officinalis Rehd. et Wils. extract feed additive.

5. The method, as recited in claim 4, wherein a step of pretreating and smashing Magnolia officinalis Rehd. et Wils. comprises naturally drying and impurity removing stems and leafs of the Magnolia officinalis Rehd. et Wils., drying for 6 h at 60° C., smashing, and sieving through a sieve with an aperture of 1.0-1.2 mm.

6. The method, as recited in claim 4, wherein an amount of the added ethanol solution is 15-20 ml ethanol solution per gram Magnolia officinalis Rehd. et Wils., wherein a concentration of ethanol in the ethanol solution is 75 wt %-80 wt %.

7. The method, as recited in claim 5, wherein an amount of the added ethanol solution is 15-20 ml ethanol solution per gram Magnolia officinalis Rehd. et Wils., wherein a concentration of ethanol in the ethanol solution is 75 wt %-80 wt %.

8. The method, as recited in claim 4, wherein while ultrasonically extracting, an ultrasonic frequency is 125-130 KHz.

9. The method, as recited in claim 7, wherein while ultrasonically extracting, an ultrasonic frequency is 125-130 KHz.

10. The method, as recited in claim 4, wherein while ultrasonically extracting, moisture is distilled simultaneously for collecting a distillate.

11. The method, as recited in claim 9, wherein while ultrasonically extracting, moisture is distilled simultaneously for collecting a distillate.

12. The method, as recited in claim 4, wherein in the step (2), acetic acid is added to adjust a PH value of solution to 6.5, and in the step (3), saturated NaOH is added to adjust a PH value of solution to 8.5.

13. The method, as recited in claim 11, wherein in the step (2), acetic acid is added to adjust a PH value of solution to 6.5, and in the step (3), saturated NaOH is added to adjust a PH value of solution to 8.5.

14. The method, as recited in claim 4, wherein before spraying and drying, the step (4) further comprising steps of: eluting the first extractum with silica gel column chromatography gradient which takes petroleum ether/ethyl acetate as an eluent with a ratio of petroleum ether/ethyl acetate=10:1 and 5:1, and an eluting time of 15 min, combining eluting solutions, concentrating for obtaining a second extractum, spraying and drying the second extractum to obtain the Magnolia officinalis Rehd. et Wils. extract dry powder.

15. The method, as recited in claim 13, wherein before spraying and drying, the step (4) further comprising steps of: eluting the first extractum with silica gel column chromatography gradient which takes petroleum ether/ethyl acetate as an eluent with a ratio of petroleum ether/ethyl acetate=10:1 and 5:1, and an eluting time of 15 min, combining eluting solutions, concentrating for obtaining a second extractum, spraying and drying the second extractum to obtain the Magnolia officinalis Rehd. et Wils. extract dry powder.

16. The method, as recited in claim 4, wherein a weight ratio of the Magnolia officinalis Rehd. et Wils. extract dry power and the carrier is 1:(2-3).

17. The method, as recited in claim 14, wherein a weight ratio of the Magnolia officinalis Rehd. et Wils. extract dry power and the carrier is 1:(2-3).

18. The method, as recited in claim 15, wherein a weight ratio of the Magnolia officinalis Rehd. et Wils. extract dry power and the carrier is 1:(2-3).

19. The method, as recited in claim 4, wherein the carrier is a mixture of attapulgite and pine needle powder with a weight ratio of 1:(1-2).

20. The method, as recited in claim 18, wherein the carrier is a mixture of attapulgite and maltodextrin with a weight ratio of 1:(1-2).
